(19) United States
(12) Patent Application Publication
(10) Pub. No.: US 2018/0188243 A1
(43) Pub. Date: Jul. 5, 2018

(54) QUANTITATIVE FRET-BASED INTERACTION ASSAY

(71) Applicant: The Regents of the University of California, Oakland, CA (US)

(72) Inventors: Jiayu Liao, Carlsbad, CA (US); Jiang Ling, Riverside, CA (US); Zhehao Xiong, Riverside, CA (US)

(21) Appl. No.: 15/737,477

(22) PCT Filed: Jun. 22, 2016
(86) PCT No.: PCT/US16/38629

§ 371 Date: Dec. 18, 2017

Related U.S. Application Data
(60) Provisional application No. 62/183,179, filed on Jun. 22, 2015.

(51) Int. Cl. G01N 33/542; G01N 21/64; G01N 33/68; C12Q 1/6818; G06F 7/483; G06F 19/16; G01N 21/76; G01N 33/557

(52) U.S. Cl. CPC G01N 33/542; G01N 21/6428; G01N 33/6845; C12Q 1/6818; G01N 2021/6432; G06F 19/16; G01N 21/76; G01N 33/557; G06F 7/483

(57) ABSTRACT

The disclosure provides a FRET-based protein interaction assay that is capable of determining the dissociation constant for interactions between two proteins even if protein contaminants are present.

### CROSS REFERENCE TO RELATED APPLICATIONS

This application claims priority under 35 U.S.C. § 119 from Provisional Application Ser. No. 62/183,179, filed Jun. 22, 2015, the disclosure of which is incorporated herein by reference.

### TECHNICAL FIELD

The disclosure provides a FRET-based assay that is capable of determining the protein interaction dissociation constant (K_{d}) for interactions between two proteins or other biochemical parameters, such as K_{cat}, Kₘ, and Kᵢ even if protein contaminants are present or unpurified protein or other biomolecules.

### BACKGROUND

Protein-protein interactions have pivotal roles in most physiology processes. In the post-genomic era, genome wide studies of protein-protein interactions and structure have effectively identified physically interactive players and potential drug targets for various human diseases. One of most important parameters for describing the protein-protein interactions affinities is the dissociation constant K_{d}. K_{d} has been determined by many techniques, including surface plasmon resonance (SPR), isothermal titration calorimetry (ITC), and radio-ligand binding assay.

### SUMMARY

The disclosure provides for an innovative quantitative Forester Resonance Energy Transfer (FRET)-based protein interaction assay to determine the dissociation constant (K_{d}) between two proteins in the presence of multiple contaminant proteins, in cellular milieu and whole cell systems.

The disclosure provides a Forester Resonance Energy Transfer (FRET)-based molecule interaction method to determine a dissociation constant (K_{d}) between two molecules in the presence of one or more contaminant molecules. The method includes providing a mixture comprising an engineered first molecule comprising a FRET donor and an engineered second molecule comprising a FRET acceptor, wherein the mixture may further comprise one or more contaminant molecules; determining the absolute FRET emission signal value (Em_{FRET}); determining the maximum amount of the FRET-donor/first molecule is bound by FRET-acceptor/second molecule (EM_{FRETmax}) by using nonlinear regression and measuring the emissions from fixed concentrations of FRET donor/first molecule and varying concentrations of the FRET acceptor/second molecule by exciting the FRET pair; and determining the K_{d} for the first and second molecule, by using nonlinear regression and the formula of:

$${Em}_{FRET} = {Em}_{{FRET}_{max}} ( 1 - \frac{2 {K}_{d}}{X - A + {K}_{d} + \sqrt{{( X - A - {K}_{d} )}^{2} + 4 {K}_{d} X}} )$$

wherein, A is the total concentration of FRET donor/first molecule, and X is the total concentration of FRET acceptor/second molecule. In one embodiment, the EM_{FRET} can be determined using the formula of:

Em_{FRET}=FL_{DA}−α*FL_{DD}−β*FL_{AA}

wherein, FL_{DA} is the fluorescence emission that is measured when FRET donor is excited by a first wavelength of light and transmits the energy to the FRET acceptor, FL_{DD} is the fluorescence emission of FRET donor/first molecule when excited by a second wavelength of light, and FL_{AA} is the fluorescence emission of FRET acceptor/second molecule when excited by a third wavelength of light, α is constant determined by using free FRET donor/first molecule, and β is a constant determined by free using FRET acceptor/second molecule. In another embodiment, the FRET donor is CyPet. In yet another or further embodiment, the FRET acceptor is YPet. In another embodiment of any of the foregoing embodiments, the first and second molecule are independently selected from the group consisting of a peptide, a polypeptide, a protein, a nucleic acid molecule, a lipid, and a polysaccharide. For example, in one embodiment, the first and second molecule can each be peptides, polypeptide or proteins. In yet another example, the first molecule can comprise a nucleic acid and the second molecule can comprise a DNA binding protein. In another example, the first molecule can be an enzyme on the second molecule can be a carbohydrate, lipid or lipoprotein. In another embodiment, the first molecule and second molecule are an enzyme and its substrate, respectively. In another embodiment, the first molecule and second molecule are a receptor and its ligand, respectively. In yet another embodiment, first molecule and second molecule are an antibody and its antigen, respectively. In yet another embodiment, the first wavelength of light is between 400 to 800 nm. In one embodiment, the first molecule comprising a FRET donor comprises a fusion protein. In another or further embodiment, the second molecule comprising a FRET acceptor comprises a fusion protein. In another embodiment, the first molecule and second molecule are expressed in the same cell. In yet another embodiment, the K_{d} is determine in an intact cell. In still another embodiment, the K_{d} is determined in a disrupted cell preparation. In another embodiment, the first and second molecule are expressed and isolated and mixed with contaminant molecules. In another embodiment, the first molecule comprising a FRET donor comprises an engineered protein. In another embodiment, the second molecule comprising a FRET acceptor comprises an engineered protein. In still another embodiment, the first or second molecule comprise DNA. In another embodiment, the first or second molecule comprise lipids. In yet another embodiment, the first or second molecule comprise polysaccharides.

### DESCRIPTION OF DRAWINGS

FIG. 1A-B provides a schematic diagram of FRET-based assay for determination of protein interaction dissociation constant, K_{d}, in the presence of competitors and/or contaminants. (A) Schematic graph of fluorescence excitation and emission signals of interactive proteins, CyPetRanGAP1c and YPetUbc9, in the presence of other proteins/contaminants. (B) A formula for K_{d} determination by FRET and fluorescence signals.

FIG. 2A-B presents the fluorescence signal analysis and titration of a FRET signal. (A) The FRET signal titration with increasing concentrations of YPetUbc9. (B) Fractionations of FRET and fluorescence signal.

FIG. 3A-D presents Em_{FRET} at different concentrations of purified CyPetRanGAP1c and YPetUbc9 in the presence or absence of other proteins. (A) Graph of Em_{FRET} at 0.05, 0.1, 0.5, 1.0 μM of purified CyPetRanGAP1c with increasing concentration of purified YPetUbc9. (B) Graph of Em_{FRET} at 0.05, 0.1, 0.5, 1.0 μM of purified CyPetRanGAP1c with increasing concentration of purified YPetUbc9 in presence of 1 μg BSA. (C) Graph of Em_{FRET} at 0.05, 0.1, 0.5, 1.0 μM of purified CyPetRanGAP1c with increasing concentration of purified YPetUbc9 in presence of 1, 3, 10 μg bacterial protein extracts. (D) Graph of Em_{FRET} at 0.05, 0.1, 0.5, 1.0 μM of unpurified CyPetRanGAP1c with increasing concentration of unpurified YPetUbc9 from crude bacterial extracts.

FIG. 4A-C presents the commassie-stained SDS-PAGE gels of protein mixtures in FRET assay. (A) lane 1, 0.2 μM CyPetRanGAP1c+1 μM YPetUbc9; lane 2, 0.2 μM CyPetRanGAP1c+1 μM YPetUbc9+1 μg BSA; lane 3, 0.2 μM CyPetRanGAP1c+1 μM YPetUbc9+3 μg BSA; lane 4, 0.5 μM CyPetRanGAP1c+1 μM YPetUbc9; lane 5, 0.5 μM CyPetRanGAP1c+1 μM YPetUbc9+1 μg BSA; lane 6, 0.5 μM CyPetRanGAP1c+1 μM YPetUbc9+3 μg BSA; lane 7, 1.0 μM CyPetRanGAP1c+1 μM YPetUbc9; lane 8, 1.0 μM CyPetRanGAP1c+1 μM YPetUbc9+1 μg BSA; lane 9, 1.0 μM CyPetRanGAP1c+1 μM YPetUbc9+3 μg BSA; (B) lane 1, CyPetRanGAP1c; lane 2, YPetUbc9; lane 3, 0.2 μM CyPetRanGAP1c+1 μM YPetUbc9+1 μg E. coli supernatant; lane 4, 0.2 μM CyPetRanGAP1c+1 μM YPetUbc9+3 μg E. coli supernatant; lane 5, 0.2 μM CyPetRanGAP1c+1 μM YPetUbc9+10 μg E. coli supernatant; lane 6, 0.5 μM CyPetRanGAP1c+1 μM YPetUbc9+1 μg E. coli supernatant; lane 7, 0.5 μM CyPetRanGAP1c+1 μM YPetUbc9+3 μg E. coli supernatant; lane 8, 0.5 μM CyPetRanGAP1c+1 μM YPetUbc9+10 μg E. coli supernatant; lane 9, 1.0 μM CyPetRanGAP1c+1 μM YPetUbc9+1 μg E. coli supernatant; lane 10, 1.0 μM CyPetRanGAP1c+1 μM YPetUbc9+3 μg E. coli supernatant; lane 11, 1.0 μM CyPetRanGAP1c+1 μM YPetUbc9+10 μg E. coli supernatant; (C) lane 1, 0.2 μM CyPetRanGAP1c supernatant (not purified); lane 2, 0.5 μM CyPetRanGAP1c supernatant (not purified); lane 3, 1.0 μM CyPetRanGAP1c supernatant (not purified); lane 4, 0.2 μM YPetUbc9 supernatant (not purified); lane 5, 0.5 μM YPetUbc9 supernatant (not purified); lane 6, 1.0 μM YPetUbc9 supernatant (not purified); lane 7, 0.2 μM CyPetRanGAP1c supernatant (not purified)+1.0 μM YPetUbc9 supernatant (not purified); lane 8, 0.5 μM CyPetRanGAP1c supernatant (not purified)+1.0 μM YPetUbc9 supernatant (not purified); and lane 9, 1.0 μM CyPetRanGAP1c supernatant (not purified)+1.0 μM YPetUbc9 supernatant (not purified).

FIG. 5A-C presents Em_{FRETmax} at different concentrations of the donor CyPetRanGAP1c in the absence and presence of other proteins. (A) The maximal FRET emission is proportional to the amount of CyPetRanGAP1c in the assay. (B) Bar graph of Em_{FRETmax} at different concentrations of CyPetRanGAP1c. (C) Bar graph of K_{d} at different concentrations of CyPetRanGAP1c.

FIG. 6A-B provides for the determination of interaction affinity K_{d} by surface plasma resonance. (A) Determination of K_{d} between the fusion proteins CyPetRanGAP1c and YPetUbc9 interaction. The K_{d} is 0.182 μM. (B) Determination of K_{d} between the Aos1 and Uba2, 0.097 μM.

### DETAILED DESCRIPTION

As used herein and in the appended claims, the singular forms “a,” “and,” and “the” include plural referents unless the context clearly dictates otherwise. Thus, for example, reference to “an assay” includes a plurality of such assays and reference to “the protein” includes reference to one or more proteins or equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood by one of ordinary skill in the art to which this disclosure belongs. Although many methods and reagents similar to or equivalent to those described herein can be used in the practice of the disclosed methods and compositions, the exemplary methods and materials are now described.

All publications mentioned herein are incorporated herein by reference in their entirety for the purposes of describing and disclosing methodologies that might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in the publications that is similar to, or identical with, a term that has been expressly defined in this disclosure, the definition of the term as expressly provided in this disclosure will control in all respects.

Molecular interactions in a cell or biological system play critical roles for biological activity and life. For example, interactions between a nucleic acid and a DNA binding protein that regulates gene expression, the interaction between an enzyme and a substrate, the interaction between a cell receptor and its ligand, the interaction between a lipid and a receptor and the like. Determining the characteristics of such interactions help in understanding the biological role of such molecules as well as methods of regulating and manipulating their interactions.

For example, proteins play a critical role in cellular processes in health or disease systems. In fact, a protein rarely has a single function; essentially all cellular functions involve protein-protein interactions. Methodologies have become a core component of studying protein interactions, which aims to define protein functions. Protein interactions are inherently dynamic, and complex mixtures of stable and transient interactions, even contaminate proteins routinely co-exist and can affect the K_{d}. Thus, the study of protein interactions is not without difficulty.

Affinity-based methods for identifying interactions have grown to encompass a wide variety of techniques with the ability to study diverse biological system. The dissociation constant, K_{d}, is normally used to determine the interaction between proteins which form non-covalent bonds. The classic methods for K_{d} determinations, such as SPR, ITC, radio-ligand binding and ultracentrifugation, have their advantages. But these methods usually require tedious procedures and expensive instruments, and additionally are limited to characterization of interactions between two purified proteins.

The dissociation constant, K_{d} can be determined by many different methods, which include fluorometric, surface-plasmon resonance (SPR), ITC, radioactive labeling and ultracentrifugation, etc. These methods offer experimental convenience, but also have some disadvantages. They often require environmentally unfriendly labeling or expensive instrumentation. Further, these methods are not reliable when the tested samples comprises disrupted cell systems, whole cells, cell fractions or containment proteins. Isothermal titration calorimetry (ITC) requires relatively large amounts (i.e., micromolar range) of samples and thus might not be suitable for high affinity binding (i.e., K_{d} of about 5 nM). It also requires a relatively expensive piece of dedicated equipment. Ultracentrifugation methods can perturb the equilibrium between bound and free proteins, especially if the dissociation rates are fast, and thus the K_{d} values determined using such methods may not represent true equilibrium constants. Finally, peripheral proteins can be nonspecifically adsorbed on the test tube walls during high-speed centrifugation. The intrinsic fluorescence method requires the presence of a tryptophan moiety in the vicinity of the protein binding surface, and thus tryptophan fluorescence changes might not quantitatively reflect the degree of proteins binding. The relatively low sensitivity of the method entails the use of micromolar protein concentrations for assay.

The Foster Resonance Energy Transfer (FRET) method in general is more sensitive than the intrinsic fluorescence measurement and offers more flexibility in assay design. Although the FRET method shares some similarity in drawbacks with the intrinsic fluorescence measurement, the use of fluorescent protein can alleviate these problems. Choice of FRET pairs, allows one to tailor the sensitivity of the assay.

The methods disclosed herein utilize highly sensitive FRET pairs. An additional consideration is that the FRET pairs not negatively influence the activity of the tagged molecules. Exemplary FRET pairs include, for example, CyPet and YPet. These FRET pairs do not negatively influence the tagged molecules to any significant extent. Other FRET pairs and fluorescent molecules are known in the art, described herein and can be used in the methods and compositions of the disclosure.

As used herein, the term “fluorescent protein” refers to any protein capable of fluorescence when excited with appropriate electromagnetic radiation. This includes fluorescent proteins whose amino acid sequences are either natural or engineered. Many cnidarians use green fluorescent proteins (“GFPs”) as energy-transfer acceptors in bioluminescence. A green fluorescent protein, as used herein, is a protein that fluoresces green light, and a blue fluorescent protein is a protein that fluoresces blue light etc. GFPs have been isolated from the Pacific Northwest jellyfish, Aequorea victoria, the sea pansy, Renilla reniformis, and Phialidium gregarium. The proteins from such organisms have been cloned, sequence and engineered and are well known in the art, including their primary and tertiary sequences. For example, a variety of Aequorea-related GFPs having useful excitation and emission spectra have been engineered by modifying the amino acid sequence of a naturally occurring GFP from Aequorea victoria. (Prasher et al., Gene, 111:229-233 (1992); Heim et al., Proc. Natl. Acad. Sci., USA, 91:12501-04 (1994); U.S. Pat. No. 5,625,048, filed Nov. 10, 1994; International application PCT/US95/14692, filed Nov. 10, 1995).

The disclosure provides at least a pair of molecules (e.g., synthetic or recombinant polypeptides) that include a first fluorescent moiety and a second fluorescent moiety, one on each molecule of the pair molecules suitable for FRET. A fluorescent moiety can include a dye, a fluorescent amino acid, a fluorescent protein and the like. The molecular pairs can be any molecule that undergoes an interaction (e.g., a peptide-peptide, polypeptide-peptide, protein-protein, protein-nucleic acid, protein-lipid, protein-carbohydrate, nucleic acid-nucleic acid etc.). In some embodiments, the label comprises a fluorescent protein which is incorporated into the molecule as part of a fusion construct (e.g., a fusion protein). Fluorescent proteins may include green fluorescent proteins (e.g., GFP, eGFP, AcGFP, TurboGFP, Emerald, Azami Green, and ZsGreen), blue fluorescent proteins (e.g., EBFP, Sapphire, and T-Sapphire), cyan fluorescent proteins (e.g., ECFP, mCFP, Cerulean, CyPet, AmCyan1, and Midoriishi Cyan), yellow fluorescent proteins (e.g., EYFP, Topaz, Venus, mCitrine, YPet, PhiYFP, ZsYellow1, and mBanana), and orange and red fluorescent proteins (e.g., Kusabira Orange, mOrange, dTomato, dTomato-Tandem, DsRed, DsRed2, DsRed-Express (T1), DsREd-Monomer, mTangerine, mStrawberry, AsRed2, mRFP1, JRed, mCherry, HcRed1, mRaspberry, HcRed-Tandem, mPlum and AQ143). Other fluorescent proteins are described in the art (Tsien, R. Y., Annual. Rev. Biochem. 67:509-544 (1998); Shaner et al., Nat. Methods, 2(12):905-909, suppl. (2005); and Lippincott-Schwartz et al., Science 300:87-91 (2003)). As noted above, the molecule may be a fusion construct such as a fusion polypeptide that include a fluorescent moiety (e.g., a fluorescent protein) coupled at the N-terminus or C-terminus of a protein or polypeptide of interest. The fluorescent moiety may be coupled via a peptide linker as described in the art (U.S. Pat. No. 6,448,087; Wurth et al., J. Mol. Biol. 319:1279-1290 (2002); and Kim et al., J. Biol. Chem. 280:35059-35076 (2005), which are incorporated herein by reference in their entireties). In some embodiments, suitable linkers may be about 8-12 amino acids in length.

Typically the molecules comprising the fluorescent moieties (FRET pairs) are ligands of one another or cognates of one another (e.g., a protein receptor and a ligand, an antibody and an antigen, a nucleic acid and a binding protein, a lipid and a receptor etc.).

A molecule of the disclosure comprising a donor or acceptor moiety of a FRET pair can be chemical synthesized using, e.g., a peptide or nucleic acid synthesizer or can be recombinantly produced using techniques known in the art. Such techniques including cloning a polynucleotide sequence, e.g., encoding the polypeptide of interest such that it is operably linked to a fluorescent moiety (e.g., a fluorescent protein).

The term “polynucleotide” or “nucleic acid” refers to a polymeric form of nucleotides. By “isolated polynucleotide” is meant a polynucleotide that is no longer immediately contiguous with both of the coding sequences with which it was immediately contiguous (one on the 5′ end and one on the 3′ end) in the naturally occurring genome of the organism from which it is derived. As such, the term “isolated polynucleotide” includes, for example, a recombinant DNA, which can be incorporated into a vector, including an autonomously replicating plasmid or virus; or into the genomic DNA of a prokaryotic or eukaryotic cell or organism; or that exists as a separate molecule (e.g. a CDNA) independent of other sequences. The nucleotides of the disclosure can be ribonucleotides, deoxyribonucleotides, or modified forms thereof, and the polynucleotides can be single stranded or double stranded.

The term “operatively linked” refers to a juxtaposition wherein the components so described are in a relationship permitting them to function in their intended manner. With reference to nucleic acids that are operatively linked, each distinct nucleic acid molecule is ligated in such a way so as to encode a polypeptide that is functional for its intended purpose. For example, an expression control sequence operatively linked to a coding sequence is ligated such that expression of the coding sequence is achieved under conditions compatible with the expression control sequences.

As used herein, the term “expression control element” refers to a nucleic acid that regulates the expression of a polynucleotide to which it is operatively linked. Expression control elements are operatively linked to a nucleic acid when the expression control elements control and regulate the transcription and, as appropriate, translation of the nucleic acid. Thus, expression control elements can include appropriate promoters, enhancers, transcription terminators, a start codon (i.e., ATG) in front of a protein-encoding nucleic acid sequence, splicing signals for introns, maintenance of the correct reading frame of that gene to permit proper translation of the mRNA, and stop codons. The term “control domain” is intended to include, at a minimum, components whose presence can influence expression, and can also include additional components whose presence is advantageous, for example, leader sequences and chimeric partner sequences.

A “peptide”, “polypeptide” or “protein” generally refer to a polymer comprising amino acids. A “peptide” refers to a polymer of amino acids of about 2-30 amino acids in length. A “polypeptide” refers to a polymer comprising about 51 to several thousand amino acids in length and may be in primary linear form, include a secondary or tertiary structure and may include various biological modifiers (e.g., co-factors). A “protein” generally refers to a proteinaceous material that comprises a polymer of amino acids and has secondary, tertiary or a heteropolymeric form, and y of which can include co-factors.

The term “promoter” refers to a minimal sequence sufficient to direct transcription. Also included in the disclosure are those promoter elements that are sufficient to render promoter-dependent gene expression controllable for cell-type specific, tissue-specific, or inducible by external signals or agents; such elements may be located in the 5′ or 3′ regions of the gene. Both constitutive and inducible promoters, are included in the disclosure (see e.g., Bitter et al., 1987, Methods in Enzymology 153:516 544). For example, when cloning in bacterial systems, inducible promoters such as pL of bacteriophage-gamma, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter; CMV promoter) may be used. Promoters produced by recombinant DNA or synthetic techniques may also be used to provide for transcription of the nucleic acid sequences of the disclosure.

By “transformation” is meant a permanent or transient genetic change induced in a cell following incorporation of new DNA (i.e. DNA exogenous to the cell). Where the cell is a mammalian cell, a permanent genetic change is generally achieved by introduction of the DNA into the genome of the cell.

By “transformed cell” is meant a cell into which (or into an ancestor of which has been introduced), by means of recombinant DNA techniques, a DNA molecule encoding a fusion polypeptide or other construct comprising a fluorescent moiety has been introduced.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl2 method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransfected with DNA sequences encoding the chimeric polypeptide of the disclosure, and a second foreign DNA molecule encoding a selectable phenotype, such as the herpes simplex thymidine kinase gene. Another method is to use a eukaryotic viral vector, such as simian virus 40 (SV40) adenovirus, vaccinia virus, or bovine papilloma virus, to transiently infect or transform eukaryotic cells and express the protein. (Eukaryotic Viral Vectors, Cold Spring Harbor Laboratory, Gluzman ed., 1982). Methods of stable transfer, meaning that the foreign DNA is continuously maintained in the host, are known in the art.

Eukaryotic systems, and mammalian expression systems, allow for proper post-translational modifications of expressed mammalian proteins to occur. Eukaryotic cells which possess the cellular machinery for proper processing of the primary transcript, glycosylation, phosphorylation, and, secretion of the gene product should be used as host cells for the expression of fluorescent indicator. Such host cell lines may include but are not limited to CHO, VERO, BHK, HeLa, COS, MDCK, Jurkat, HEK-293, and WI38.

Mammalian cell systems which utilize recombinant viruses or viral elements to direct expression may be engineered. For example, when using adenovirus expression vectors, the nucleic acid sequences encoding a fluorescent construct of the disclosure may be ligated to an adenovirus transcription/translation control complex, e.g., the late promoter and tripartite leader sequence. This nucleic acid sequence may then be inserted in the adenovirus genome by in vitro or in vivo recombination. Insertion in a non-essential region of the viral genome (e.g., region E1 or E3) will result in a recombinant virus that is viable and capable of expressing the fluorescent indicator in infected hosts (see, for example, Logan & Shenk, Proc. Natl. Acad. Sci. USA, 81: 3655 3659, 1984). Alternatively, the vaccinia virus 7.5K promoter may be used (see, for example, Mackett et al., Proc. Natl. Acad. Sci. USA, 79: 7415 7419, 1982; Mackett et al., J. Virol. 49: 857 864, 1984; Panicali et al., Proc. Natl. Acad. Sci. USA 79: 4927 4931, 1982). Of particular interest are vectors based on bovine papilloma virus which have the ability to replicate as extrachromosomal elements (Sarver et al., Mol. Cell. Biol. 1: 486, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of a fluorescent polypeptide in host cells (Cone and Mulligan, Proc. Natl. Acad. Sci. USA, 81:6349 6353, 1984). High level expression may also be achieved using inducible promoters, including, but not limited to, the metallothionein IIA promoter and heat shock promoters.

For long term, high yield production of recombinant proteins, stable expression can be used. Rather than using expression vectors which contain viral origins of replication, host cells can be transformed with the cDNA encoding a fluorescent polypeptide of the disclosure controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used including, but not limited to, the herpes simplex virus thymidine kinase (Wigler et al., Cell, 11: 223, 1977), hypoxanthine-guanine phosphoribosyltransferase (Szybalska and Szybalski, Proc. Natl. Acad. Sci. USA, 48:2026, 1962), and adenine phosphoribosyltransferase (Lowy et al., Cell, 22: 817, 1980) genes can be employed in tk-, hgprt- or aprt-cells respectively. Also, antimetabolite resistance can be used as the basis of selection for dhfr, which confers resistance to methotrexate (Wigler et al., Proc. Natl. Acad. Sci. USA, 77: 3567, 1980; O'Hare et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin et al., J. Mol. Biol., 150:1, 1981); and hygro, which confers resistance to hygromycin (Santerre et al., Gene, 30: 147, 1984) genes. Recently, additional selectable genes have been described, namely trpB, which allows cells to utilize indole in place of tryptophan; hisD, which allows cells to utilize histinol in place of histidine (Hartman and Mulligan, Proc. Natl. Acad. Sci. USA, 85:8047, 1988); and ODC (ornithine decarboxylase) which confers resistance to the ornithine decarboxylase inhibitor, 2-(difluoromethyl)-DL-ornithine, DFMO (McConlogue, In: Current Communications in Molecular Biology, Cold Spring Harbor Laboratory, ed., 1987).

The disclosure provides a method of determining the dissociation constant between two molecules (e.g., two polypeptides) in a non-pure environment. By “non-pure” means an environment that comprises contaminants such as other proteins, polypeptides, peptides, lipids, carbohydrates, nucleic acids, organelles, combinations of any of the foregoing etc. The method can be used in vivo, in situ or in vitro. In one embodiment, the method is performed in a disrupted cell preparation. In another embodiment, the method is performed in a system wherein a plurality of different molecules (e.g., proteins or polypeptides) are introduced. In any of the foregoing methods, a first molecule comprises a donor fluorescent moiety and a second molecule comprises an acceptor fluorescent moiety, wherein the first and second molecules are binding partners or cognates and wherein the fluorescent donor and acceptor can undergo FRET.

In one embodiment, a first fusion construct comprises a donor moiety of a FRET pair linked to a first molecule cognate of a binding pair and a second fusion construct comprises an acceptor moiety of a FRET pair linked to a second molecule cognate of a binding pair are expressed in a cell. The cell may be assayed for fluorescent and FRET intact or disrupted and their K_{d} calculated as described herein. In another embodiment, a first fusion protein comprising a donor moiety of a FRET pair linked to a first polypeptide cognate of a binding pair is expressed an isolated. A second fusion protein comprising an acceptor moiety of a FRET pair linked to a second polypeptide cognate of a binding pair are expressed and isolated. In this latter embodiment, the first and second fusion proteins are mixed in a system comprising one or more contaminates (e.g., non-binding proteins, polypeptide, or peptides; or competitive binding agents). The K_{d} of the first and second fusion proteins are then calculated. It will be readily apparent that a K_{d} determination can be used to calculate Kₘ. For example, the dissociation constant is measure of affinity, with higher values indicating lower affinity: Km=(k₋ₖ+k₂)/k1 and Kd=k₋₁/k₁. If k₂=0, then Kₘ=K_{d}. Because, for most enzymes, k₂ is relatively small compared to k₋₁, the Kₘ value is often close to the K_{d} value.

To implement FRET-based technology for K_{d} determination, two data sets are used. First, a data set is calculated that differentiate and quantify fluorescence signal of FRET from other direct fluorescence signals of donor and acceptor at the excitation wavelength. Second, a conversion of fluorescence signals to corresponding concentrations of bound partners, donor and acceptor proteins is obtained.

If one defines the molecule (e.g., polypeptide) interaction as being: P1+P2⇄P1−P2, the K_{d} can be calculated as:

$${K}_{d} = \frac{{{[ donorP 1 ]}_{free} [ acceptorP 2 ]}_{free}}{[ donorP 1 \cdot acceptorP 2 ]} = \frac{{{[ donorP 1 ]}_{free} [ acceptorP 2 ]}_{free}}{{[ acceptorP 2 ]}_{bound}} ,$$

which can be re-arranged as:

$${[ acceptorP 2 ]}_{bound} = \frac{[ acceptorP 2 ] bound {max [ acceptorP 2 ]}_{free}}{{K}_{d} + {[ acceptorP 2 ]}_{free}}$$

Where [acceptorP2]bound max is the theoretical maximal acceptorP2 concentration that is bound to the donorP1 concentration, and [accpetorP2]_{free} is the free acceptorP2 concentration. [acceptorP2]_{bound} is proportional to the FRET signal of bound proteins. The method then differentiates the acceptor emission signal into three fractions: FRET emission (Em_{FRET}), acceptor direct emission, and donor direct emission when excited at the donor excitation wavelength: Em_{FRET}=(Emₜₒₜₐₗ)−donorP1(cont)−acceptorP2(cont). The direct emission of the donor at the acceptor emission wavelength is proportional to its emission wavelength at the donor emission wavelength at ratio factor α, while the direct emission of the acceptor at the acceptor emission wavelength is proportional to its emission wavelength at the acceptor emission wavelength when excited at the acceptor excitation wavelength with a ratio factor β. Thus, the method can include determining the absolute Em_{FRET}, by determining the ratio constants α and β. α and β are determined by a dilution series of the donor fluorophore-labeled polypeptide; exciting at the excitation wavelength and measuring at the emissions wavelength for the donor and the acceptor fluorescent moiety; dividing the donor fluorescence emission value (FL_{DD}) by the emission at the acceptor wavelength to obtain the ratio constant, α. This constant is an estimate of unquenched donor to the total emission at the acceptor wavelength when excited at donor excitation wavelength. In a second series of experiments, a dilution series of the acceptor fluorophore is prepared. After exciting the acceptor at the donor wavelength and the acceptor wavelength, the emissions of acceptor at the emission wavelength is obtained for each. Dividing the fluorescence emission when excited at donor excitation wavelength by its emission the acceptor excitation wavelength (FL_{AA}) to obtain the ratio constant, β. Thus, Em_{FRET}=(Emₜₒₜₐₗ)−(α*FL_{DD})−(β*FL_{AA}), where FL_{DD} is fluorescence signal of donor when excited at donor wavelength and FL_{AA} is fluorescence signal of acceptor when excited at acceptor wavelength.

After the intensity of Em_{FRET} at each specific condition is calculated, the datasets of Em_{FRET} and total concentration of acceptorP2 ([acceptorP2]ₜₒₜₐₗ) are fitted using, e.g., Prism 5 (GraphPad Software), to derive values for Em_{FRET} max and K_{d}. The values of [acceptorP2]ₜₒₜₐₗ are put into X-series, and the intensities of Em_{FRET} that are determined in triplicate at each [acceptorP2]ₜₒₜₐₗ were put into Y-series. A nonlinear regression method is selected and a customized equation is created to fit the datasets:

$$Y = \frac{EmFRET max - 2 * EnFRET max * {K}_{d}}{( X - A + {K}_{d} + sqrt ( sqr ( X - A - {K}_{d} ) + 4 * {K}_{d} * X ) )}$$

The initial values of the parameters Em_{FRET}, K_{d} and A are set to 1.0. The default constraint Em_{FRETmax} value must be greater than 0. The A constant is equal to the concentration added to the system (e.g., 0.05, 0.1, 0.5, and 1.0 μM). The results are reported as mean±SD.

Thus, determining the K_{d} for the first and second protein, by using nonlinear regression and the formula of:

$${Em}_{FRET} = {Em}_{{FRET}_{max}} ( 1 - \frac{2 {K}_{d}}{X - A + {K}_{d} + \sqrt{{( X - A - {K}_{d} )}^{2} + 4 {K}_{d} X}} )$$

wherein, A is the total concentration of FRET donor/first protein, and X is the total concentration of FRET acceptor/second protein.

The methods of the disclosure were demonstrated using small ubiquitin-related modifiers. Small ubiquitin-related modifiers (SUMOs) are ubiquitin-like polypeptides that are covalently conjugated to target proteins as one type of major protein post-translational modification. Modifications by the SUMO family of peptides regulate many important biological processes, such as nuclear transport, transcription, DNA repair and other stress responses, the cell cycle, and apoptosis. SUMO conjugation occurs through an enzymatic cascade, involving E1-activating enzyme, E2-conjugating enzyme and E3 protein ligases. The SUMO E1 activating enzyme consists of a heterodimeric complex, Aos1 and Uba2, and initiates the conjugation process by adenylating the SUMO C-terminus with ATP and Mg²⁺. A thioester bond forms between the active-site cysteine residue of Uba2 and the C-terminal glycine residue of SUMO. This reaction is followed by a transesterification of SUMO to a cysteine residue of Ubc9, the E2 conjugating enzyme. The E3 ligases facilitate most SUMOylation under physiological conditions by recruiting E2-SUMO and substrate into a complex to promote specificity in vivo and stimulating SUMO conjugate to lysine residues of substrates under physiological conditions. Several SUMO E3 protein ligases have been described. For example, members of the Siz/PIAS family contain an RING-finger-like domain (SP-RING domain). RanBP2 has a domain called the internal repeat (IR) domain. Other SUMO ligase include histone deacetylase 4 (HDAC4), KRAB-associated protein1 (KPA1), Pc2 and Topors.

Disclosed herein, are methods directed to single step quantitative FRET assay that allowed for the determination of the disassociation constant, K_{d}, of the SUMO E2 conjugating enzyme, Ubc9, and the substrate, RanGAP1c. The methods disclosed herein comprise a highly efficient FRET pair (e.g., CyPet and YPet) to fuse with RanGAP1c and Ubc9. The results using the methods of the disclosure indicate that K_{d} mainly depends on the fluorescence signals which come from the interaction of tagged proteins. The methods disclosed herein can quantitatively determine the RanGAP1c and Ubc9 interaction affinity even in the presence of multiple contaminate proteins. For example, without BSA or E. coli BL21 lysates, CyPet-RanGAP1c ranging from 0.05 to 1.0 μM was found to have a K_{d} value of 0.102±0.008 μM using the methods of the disclosure, while in the presence of BSA, the K_{d} value was found to be 0.102±0.006 μM for various concentrations of CyPet-RanGAP1c ranging from 0.05 to 1.0 μM; and further in the presence of E. coli BL21 lysates, the K_{d} value was found to be 0.098±0.007 μM for various concentrations of CyPet-RanGAP1c ranging from 0.1 to 1 μM. Moreover, from directly extracted CyPet-RanGAP1c and YPetUbc9 proteins from E. coli BL21 lysates, the crude proteins were found to have a K_{d} value of 0.099±0.002 μM. The results are very consistent and in good agreement with the K_{d} values determined by using a traditional SPR method (97 and 182 nM for the CyPet-RanGAP1c and YPet-Ubc9 or the RanGAP1c and Ubc9, respectively). A previous ITC study also showed high affinity interaction between the Ubc9 and RanGAP1c (K_{d}˜0.49 μM).

Additionally, results using the methods of the disclosure were further validated by using a SPR method. The results from the SPR method agreed with the results from the methods disclosed herein. Compared with SPR, the FRET-based method disclosed herein is better able to determine a protein-protein interaction, especially if an enzyme is involved. SPR is not well particularly well suited to study protein-protein interactions as there is always a potential orientation problem associated with immobilizing the protein for the SPR assay. Also kinetic SPR data can be quite complex due to various effects, including mass transfer effect, rebinding effect, and nonspecific binding to the sensor chip, and thus careful mathematical analysis is needed to obtain meaningful parameters. Further, the SPR method for K_{d} determination might not be valid when a simple Langmuir-type binding model fails to apply. Additional drawbacks were found when digesting his-tag by thrombin in SPR assay. In order to perform the digestion reaction, the reaction temperature had to be maintained around 16° C. However, this temperature negatively influenced the activities of CyPetRanGAP1c and YPetUbc9. When BiacoreX100 was used to generate the data, the run could only be done with one concentration, and the relative protein interaction response took 40 min. So if the BiacoreX100 is used with many samples or replicates the overall reaction time could exceed 12 hours. As enzymatic activity is labile, the results would likely be inconsistent. Alternatively, six concentrations and three time repeats using the methods disclosed herein could take less than 1 hour. Moreover, as EQ. 6 takes into account any potential orientation problems.

The high-affinity interaction of RanGAP1c and Ubc9 may provide explanations of how Ubc9 mediates SUMO conjugation to RanGAP1c directly and without the help of E3. The results further support the hypothesis that Ubc9 can mediate SUMO conjugation directly. The crystal structure of RanGAP1c-Ubc9 shows interactions between two molecules that provide the molecular basis for recognition of the RanGAP1c Ψ-K-X-D/E consensus motif. The interface can be described in two parts. One is between RanGAP1c helices H and F and Ubc9 surfaces emanating mainly from helix C. The second part includes interactions between the consensus RanGAP1c sumoylation motif (-LKSE-) and Ubc9 surfaces that include the catalytic cysteine, strands 6 and 7, and the loop preceding helix C. These interactions likely lead to an increased binding and more effective SUMO transfer. The experiments presented herein demonstrate the high-affinity interaction of RanGAP1c-Ubc9 by using the quantitative FRET based method disclosed herein.

The consistent affinity results of the K_{d} determination at nanomolar range not only shows that the method of the disclosure is not only accurate and reliable at various concentrations of the interactive partners, but also sensitive at high affinity nanomolar levels. In contrast, traditional radio-labeled protein binding assays to determine K_{d} requires a range of at least 100-fold of labeled ligand to predict maximum binding. The FRET-based K_{d} determination approach taught herein accurately determines K_{d} at ratios 0.67-40 fold of the binding partners of the RanGAP1c-Ubc9. Other approaches for K_{d} determination, such as SPR or isothermal titration calorimetry, require multiple steps and special instruments and often give large variations. While the FRET assay has become more popular in biochemical and cell biology studies, the quantitative FRET method disclosed herein is a notable advance over current technology by allowing for the determination of a K_{d} in the presence of contaminant proteins, thereby providing reliable quantitative results at a fraction of the cost versus standard FRET assays.

Accordingly, the methods disclosed herein can efficiently monitor protein interactions in real time regardless of the size of substrates. Further, the methods disclosed herein can easily be developed to use a high-throughput technique.

The following examples are intended to illustrate but not limit the disclosure. While they are typical of those that might be used, other procedures known to those skilled in the art may alternatively be used.

### Examples

DNA Constructs.

The open reading frames of CyPet and YPet were amplified by PCR with primers containing NheI-SalI sites. The size of PCR products were 729 and 729 bp, respectively. RanGAP1c and Ubc9 were amplified by PCR with primers containing SalI-NotI sites. All these four genes were cloned into pCRII-TOPO vector (Invitrogen). The fragments encoding RanGAP1c and Ubc9 were extracted after a digestion by SalI-NotI and inserted into pCRII-CyPet or pCRII-YPet that had been linearized by cutting with SalI and NotI. After the sequences were confirmed by sequencing, the cDNAs encoding CyPetRanGAP1c and YPetUbc9 were cloned into the NheI-NotI sites of pET28(b) vector (Novagen).

Protein Expression, Purification and Concentration Determination.

BL21(DE3) Escherichia coli cells were transformed with pET28(b) vectors encoding CyPetRanGAP1c or YPetUbc9. The transformed bacteria were plated on LB agar plates containing 50 μg/mL kanamycin, and single colonies were picked up and inoculated in 2×YT medium to an optical density at 600 nm of 0.5-0.8. The expression of polyhistidine-tagged recombinant proteins was induced with 0.2 mM isopropyl β-D-thiogalactoside. Bacterial cells were collected by centrifugation at 6,000 rpm for 10 min, re-suspended in binding buffer (20 mM Tris-HCl, pH 7.4, 500 mM NaCl and 5 mM imidazole), and sonicated with an ultrasonic liquid processor (Misonix). Cell lysates containing recombinant proteins were cleared by centrifugation at 35,000 g for 30 min. The polyhistidine-tagged recombinant proteins were then purified from bacterial lysates with Ni²⁺-NTA agarose beads (QIAGEN) and washed by three different washing buffers (washing buffer 1 contained 20 mM Tris-HCl, pH 7.4, 300 mM NaCl; washing buffer 2 contained 20 mM Tris-HCl, pH 7.4, 1.5 M NaCl, and 5% Triton X-100; and washing buffer 3 contained 20 mM Tris-HCl, pH 7.4, 500 mM NaCl, and 20 mM imidazole). The products were then eluted by the addition of elution buffer (20 mM Tris-HCl, pH 7.4, 200 mM NaCl, and 250 mM imidazole). The proteins were purified by dialysis using a dialysis buffer (20 mM Tris-HCl, pH 7.4, 50 mM NaCl, and 1 mM DTT). The purity of proteins was confirmed by SDS-PAGE and Coomassie blue staining. Concentrations were determined by using a Coomassie Plus Protein Assay (Thermo Scientific).

FRET Measurements.

Four different mixtures for the protein-protein interaction were measured. Mixture 1: in a total volume of 60 μL, recombinant CyPetRanGAP1c and YPetUbc9 proteins were incubated and mixed at room temperature in the Tris buffer (20 mM Tris-HCl, pH 7.5, 50 mM NaCl, DTT 1 mM) to a total volume of 60 μL. The final concentrations of CyPetRanGAP1c were fixed at 0.05, 0.1, 0.5, 1.0 μM; the final concentrations of YPetUbc9 were varied from 0 to 4 μM.

Mixture 2: in a total volume of 60 μL, contaminate proteins that were extracted from wild-type BL21(DE3) E. coli, were added to a mixture comprising CyPetRanGAP1c and YPetUbc9 proteins. The concentration of contaminate proteins was 1.0, 3.0, 10.0 μg in the Tris buffer (20 mM Tris-HCl, pH 7.5, 50 mM NaCl, and DTT 1 mM). The final concentrations of CyPetRanGAP1c were fixed to 0.1, 0.5, 1 μM; the final concentrations of YPetUbc9 were varied from 0 to 4 μM.

Mixture 3: in a total volume of 60 μL, pure BSA was added to a mixture comprising CyPetRanGAP1c and YPetUbc9 proteins. The concentration of BSA was 1 μg in the Tris buffer (20 mM Tris-HCl, pH 7.5, 50 mM NaCl, and DTT 1 mM). The final concentrations of CyPetRanGAP1c were fixed to 0.05, 0.1, 0.5, 1.0 μM; the final concentrations of YPetUbc9 were varied from 0 to 4 μM.

Mixture 4: CyPetRanGAP1c and YPetUbc9 were extracted from BL21(DE3) E. coli without purification. The concentration of CyPetRanGAP1c and YPetUbc9 were measured by a fluorescence standard curve. The final concentrations of CyPetRanGAP1c were fixed to 0.05, 0.1, 0.5, 1.0 μM and the final concentrations of YPetUbc9 were varied from 0 to 4 μM.

After mixing thoroughly, the four different mixtures (Mixture 1-4) were examined with a fluorescence multi-well plate reader FlexstationII384 (Molecular Devices, Sunnyvale, Calif.). The fluorescence emission signals at 475 and 530 nm were collected under the excitation wavelength at 414 nm with a cutoff filter at 455 nm. Another fluorescence emission signal at 530 nm was collected at the excitation wavelength at 495 nm with a cutoff filter at 515 nm. The experiments were repeated three times, and the average values of fluorescence were recorded under each specific condition.

Standard Curve for CyPetRanGAP1c and YPetUbc9.

The recombinant proteins CyPetRanGAP1c and YPetUbc9 were incubated at 37° C. in Tris buffer (20 mM Tris-HCl, pH 7.5, 50 mM NaCl, DTT 1 mM), and then added (60 μL) to each well of a 384-well black/clear plate. After excitation at 414 nm, the emission signals of CyPetRanGAP1c at 475 nm were collected for samples containing protein that varied from 0 to 2 μM. After excitation at 475 nm, the emission samples for YPetUbc9 at 530 mm were collected for samples containing protein that varied from 0 to 2 μM. For measurements of raw CyPetRanGAP1c and YPetUbc9 protein concentrations, BL21 bacteria lysates were used as a control.

Fluorescence Spectrum Analysis of FRET.

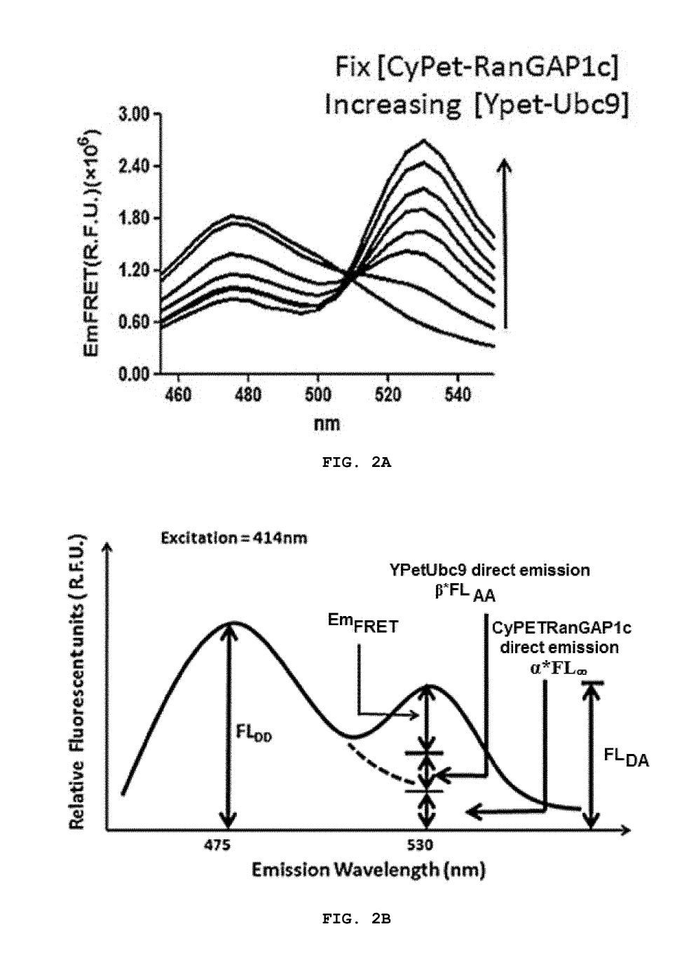

Emission peaks at 475 (FL_{DD}) and at 530 nm (Emₜₒₜₐₗ) were obtained when the mixture was excited at 414 nm (see FIG. 2B). An emission peak at 530 nm (FL_{AA}) was obtained when the mixture was excited at 495 nm. When a mixture of CyPetRanGAP1c and YPetUbc9 was excited at 414 nm, nearly all of the emission intensity at 475 nm was the direct emission of CyPetRanGAP1c after energy transfer to YPetUbc9. The emission intensity at 530 nm consisted of three components: the direct emission of CyPetRanGAP1c, the sensitized emission of YPetUbc9 (Em_{FRET}), and the direct emission of YPetUbc9. Since Em_{FRET} is proportional to the amount of YPetUbc9 bound to CyPetRanGAP1c, the relationship between Em_{FRET} and the bound concentration of YPetUbc9 is derivable.

To determine the absolute Em_{FRET}, two series of experiments were conducted to identify the ratio constants α and β. The first series of experiments were to determine the ratio constant of a. A dilution series of the donor fluorophore-labeled polypeptide (e.g., CyPetRanGAP1c) was prepared at concentrations of 1.0, 0.5, 0.1 and 0.05 μM. After exciting at 414 nm, the emissions for the donor polypeptide (e.g., CyPetRanGAP1c) were determined at 475 and 530 nm. Dividing the 475 nm fluorescence emission value (FL_{DD}) by the emission at 530 nm yielded the ratio constant, a. This constant is an estimate of unquenched donor (e.g., CyPetRanGAP1c) to the total emission at 530 nm when excited at 414 nm. In the second series of experiments, a dilution series of the acceptor fluorophore (e.g., YPetUbc9) was prepared at concentrations of 4.0, 3.0, 2.0, 1.0, 0.5, and 0.2 μM. After exciting at 414 or 495 nm, the emissions of acceptor (e.g., YPetUbc9) were determined at 530 nm. Dividing the fluorescence emission at 530 nm when excited at 414 nm by its emission at 530 nm when excited at 495 nm (FL_{AA}) yielded the ratio constant, β.

Data Processing and K_{d} Determination.

After the intensity of Em_{FRET} at each specific condition was calculated based as described above, the datasets of Em_{FRET} and total concentration of YPetUbc9 ([YPetUbc9]ₜₒₜₐₗ) were fitted by Prism 5 (GraphPad Software) to derive values for Em_{FRET} max and K_{d}. In principle, the values of [YPetUbc9]ₜₒₜₐₗ were put into X-series, and the intensities of Em_{FRET} that were determined in triplicate at each [YPetUbc9]ₜₒₜₐₗ were put into Y-series. A nonlinear regression method was selected and a customized equation was created to fit the datasets:

$${[ acceptorP 2 ]}_{bound} = \frac{[ acceptorP 2 ] bound {max [ acceptorP 2 ]}_{free}}{{K}_{d} + {[ acceptorP 2 ]}_{free}} \qquad EQ . 1$$

The initial values of the parameters Em_{FRET}, K_{d} and A are set to 1.0. The default constraint Em_{FRETmax} value must be greater than 0. The A constant is equal to the concentration added to the system (0.05, 0.1, 0.5, and 1.0 μM). The results are reported as mean±SD.

K_{d} Determination of the Non-Covalent RanGAP1c and Ubc9 Interaction by SPR.

His-tagged YPetUbc9 and CyPetRanGAP1c or His-tagged ubc9 and RanGAP1c were dialyzed overnight in running buffer (10 mM HEPES, 150 mM NaCl, 50 μM EDTA, 0.005% Tween20 pH7.4) in order to ensure that the tested conditions were the same. All analyses of interaction between CyPetRanGAP1c and YPetUbc9, or RanGAP1c and Ubc9 were performed on BIAcore X100 system equipped with NTA sensor chips (BIAcore AB, Uppsala, Sweden) at a flow rate of 30 μL/min. For immobilization of proteins, the chip was treated with 500 μM NiCl₂ in running buffer for 1 min. Purified YPetUbc9 (100 ng/mL) or purified Ubc9 protein (200 ng/mL) were then injected for 120 s and stabilized for 120 s. Next, thrombin-digested CyPetRanGAP1c protein (50˜160 μg/mL) or thrombin-digested RanGAP1c protein (10˜40 μg/mL) was injected for 120 s and disassociated for 10 min. To continuously monitor the nonspecific background binding of samples to the NTA surface, CyPetRanGAP1c and RanGAP1c proteins were injected into a control flow cell without NiCl₂ treatment and YPetUbc9/Ubc9 proteins. After determining the concentration of CyPetRanGAP1c and YPetRanGAP1c, or RanGAP1c and RanGAP1c, the NTA sensor chip was regenerated by the addition of regeneration buffer (10 mM HEPES, 150 mM NaCl, 350 mM EDTA, 0.005% Tween 20, pH 8.3). Another concentration was determined after first treating with NiCl₂, and immobilizing YPetUbc9 or Ubc9 on the chip. All measurements were performed at 25 CC in running buffer. Data were analyzed with BIAcore X100 evaluation software ver.1.0 (BIAcore).

The Design of Quantitative FRET-Based Approach for K_{d} Determination of Ubc9-RanGAP Interaction in the Presence of Other Proteins.

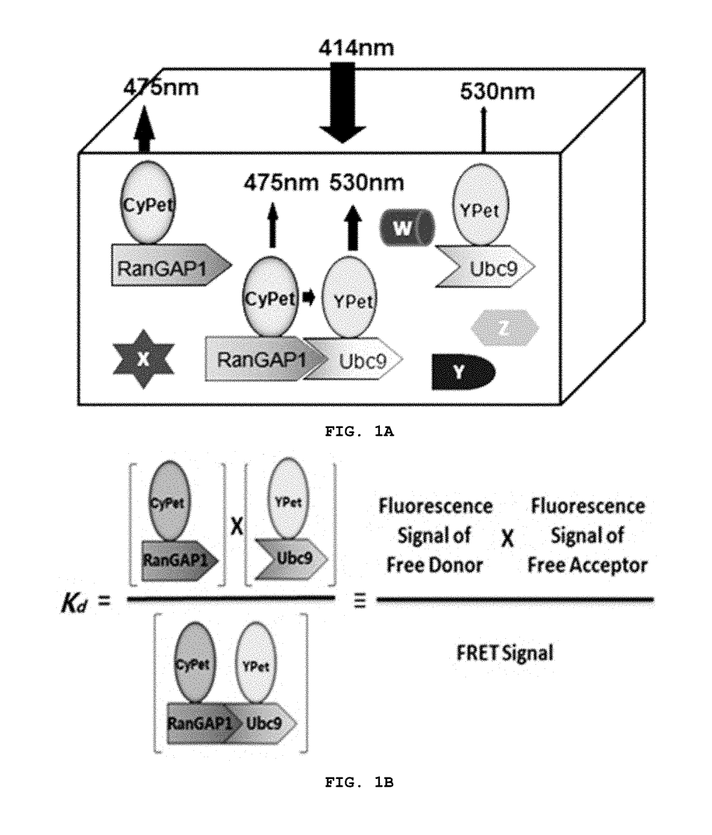

To obtain the dissociation constant, K_{d} data in tough environments, RanGAP1c and Ubc9 were chosen for determining their dissociation constant by quantitative FRET-based approach. The measurement was carried out following the Em_{FRET} signal, using FRET assay developed by Song et al. (Ann Biomed Eng 39(4):1224-34 (2011)). The Em_{FRET} was tested by injecting CyPet tagged with RanGAP1c, and YPet tagged with Ubc9, into 384 well black/clear plates. The concentration of CyPetRanGAP1c was fixed and the concentration of YPetUbc9 was titrated until the Em_{FRET} signal reached a saturating amount. When RanGAP1c and Ubc9 interact with each other, the distance of CyPet and YPet was fit for fluorescence resonance energy transfer. If the mixture was excited at 414 nm, the excitation wavelength of the donor (CyPet) there was an emission signal at 530 nm, the emission wavelength of the acceptor (YPet). This process depended on the interaction between CyPetRanGAP1c and YPetUbc9, even though there were different kinds of contaminate protein in the assay (see FIG. 1A).

In the presence of different contaminate proteins assay, the binding of two proteins can be analyzed by a simple Em_{FRET} signal following Equation 2:

RanGAP1c·Ubc9 [symbol] RanGAP1c+Ubc9 (EQ. 2)

After RanGAP1c and Ubc9 are tagged with CyPet and YPet, respectively, the dissociation constant, K_{d}, can be defined as shown in FIG. 1B according to Equation 3:

$${K}_{d} = \frac{{{[ CyPetRanGAP 1 c ]}_{free} [ YPetUbc 9 ]}_{free}}{[ CyPetRanGP 1 c \cdot YPetUBC 9 ]} = \frac{{{[ CyPetRanGAP 1 c ]}_{free} [ YPetUbc 9 ]}_{free}}{{[ YPetUbc 9 ]}_{bound}} \qquad ( EQ . 3 )$$

Equation 3 can be re-arranged as Equation 4:

$${[ YPetUbc 9 ]}_{bound} = \frac{[ YPetUbc 9 ] bound {max [ YPetUbc 9 ]}_{free}}{{K}_{d} + {[ YPetIbc 9 ]}_{free}} \qquad ( EQ . 4 )$$

Where [YPetUbc9]bound max is the theoretical maximal YPetUbc9 concentration that is bound to the CyPetRanGAP1c concentration, and [YPetUbc9]_{free} is the free YPetUbc9 concentration. [YPetUbc9]_{bound} is proportional to the FRET signal of bound proteins. (Eq. 4) can be converted into (Eq. 6) using the relationship expressed in (Eq. 5):

$$\begin{matrix} \frac{{[ YPetUbc 9 ]}_{bound}}{{[ YPetUbc 9 ]}_{{bound}_{max}}} = \frac{{Em}_{FRET}}{{Em}_{{FRET}_{max}}} & ( Eq . 5 ) \\ {Em}_{FRET} = {Em}_{{FRET}_{max}} ( 1 - \frac{2 {K}_{d}}{X - A + {K}_{d} + \sqrt{{( X - A - {K}_{d} )}^{2} + 4 {K}_{d} X}} ) & ( Eq . 6 )\end{matrix}$$

where Em_{FRET} is the absolute FRET signal and Em_{FRETmax} is the absolute FRET signal when the maximum amount of YPetUbc9 is bound by CyPetRanGAP1c. A is the total concentration of CyPetRanGAP1c ([CyPetRanGAP1c]ₜₒₜₐₗ), X as the concentration of total YPetUbc9 ([YPetUbc9]ₜₒₜₐₗ).

FRET Assay of YPetUbc9-CyPetRanGAP1c and Three Fraction Analysis of Fluorescence Signals at Acceptor Emission Wavelength.

In order to remove background, the plate was first scanned by itself prior to measuring the emission signals of the protein mixture comprising CyPetRanGAP1c and YPetUbc9. The blank plate was excited at 414 nm, emission signals were collected at 475 and 530 nm; the blank plate was excited at 495 nm and the emission signal at 530 nm was determined to determine a background value (see below). When the concentration of CyPetRanGAP1c was fixed, the FRET signal increased as more YPetUbc9 was added (see FIG. 2A).

To obtain an absolute FRET signal (Em_{FRET}) for the K_{d} measurement, the direct emissions at 530 nm for the donor (CyPet) and acceptor (YPet) first need to be determined and excluded from the total emission at 530 nm. The direct emission of the unquenched donor CyPet at 530 nm is proportional to its emission at 475 nm when excited at 414 nm with a ratio factor of α (α*FL_{DD}, FL_{DD} is the fluorescence emission of CyPet at 475 nm when excited at 414 nm), while the direct emission of YPet at 530 nm is proportional to its emission at 530 nm when excited at 495 nm with a ratio factor of β (β*FL_{AA}, FL_{AA} is the fluorescence emission of YPet at 530 nm when excited at 495 nm) (see FIG. 2B). Therefore the FRET emission signal of YPet (Em_{FRET}) can be determined by (Eq. 7):

Em_{FRET}=FL_{DA}−α*FL_{DD}−β*FL_{AA} (EQ. 7)

where the ratio constants α and β were first experimentally determined as 0.334±0.003 and 0.014±0.002, using free CyPetRanGAP1c and YPetUbc9, respectively.

The emission intensity at 530 nm consists of three components: the direct emission of CyPet, the direct emission of YPet and the emission of the FRET signal (Em_{FRET}) (see FIG. 2B). In the FRET assay, the mixture of CyPetRanGAP1c and YPetUbc9 was excited at 414 nm, and two emission signals at 475 nm (FL_{DD}) and 530 nm (FL_{DA}) were determined (see FIG. 2B). When excited at 414 nm, fluorescence emissions at 475 nm were from the emission of unquenched CyPet (FL_{DD}) and the direct emission of YPet at 475 nm (which is very small, <2.6% of CyPet emission and therefore can be ignored). When excited at 414 nm and 495 nm, the emissions of CyPetRanGAP1c at 475 nm and YPetUbc9 at 530 nm, respectively were determined. The FRET emission signal of YPetUbc9 can be calculated by subtracting the above two signals with the ratios α and β from the total emission at 530 nm.

In our previous studies, we analyzed the protein interaction affinity of CyPet-SUMO1 and YPetUbc9 by the quantitative FRET assay. The FRET-based K_{d} measurement provides reliable K_{d} values and has several advantages over other standard K_{d} measurement methods, such as a radiolabeled ligand binding assay, SPR or isothermal titration calorimetry. The SUMO substrate and E2, RanGAP1c and Ubc9 are important in the SUMOylation pathway. Also the SUMOylation pathway works without E3 in vitro. The interaction affinity between RanGAP1c and Ubc9 was therefore investigated in order to understand the thermodynamics of why the conjugation takes place without E3 by the above methods.

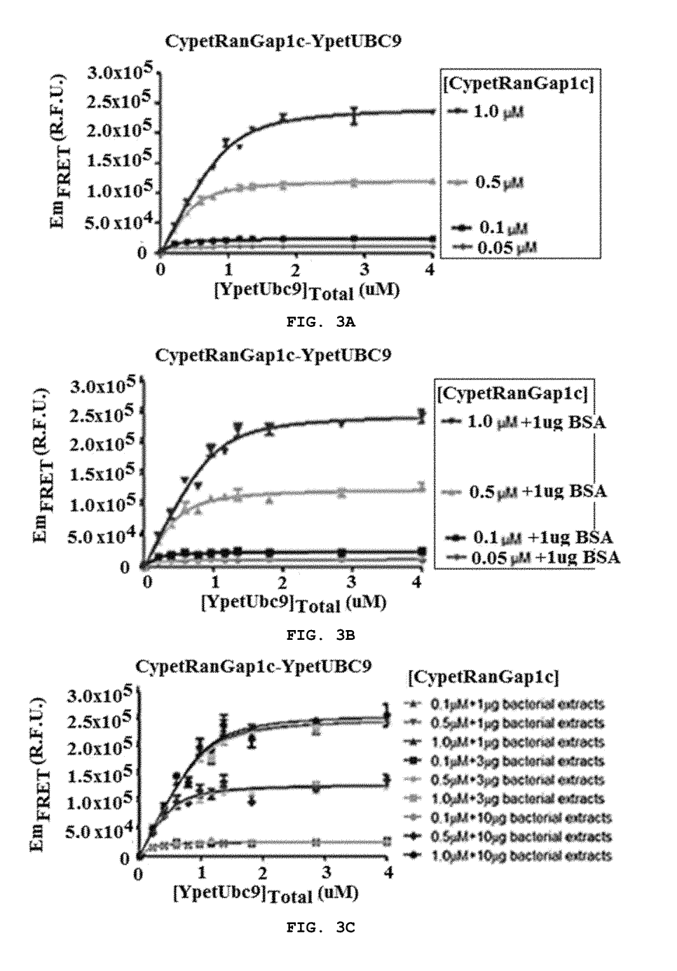

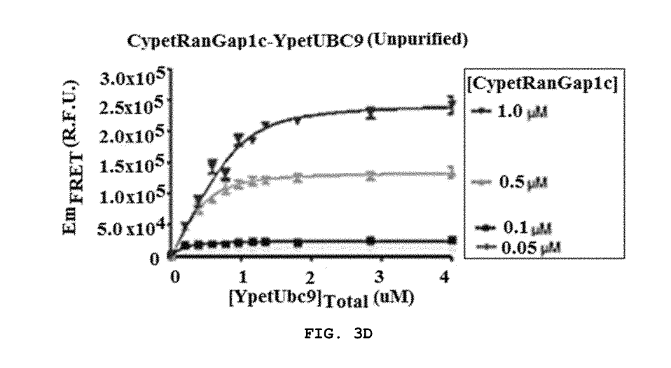

The sensitivity of the FRET assay was tested at different concentrations of CyPetRanGAP1c. FIG. 2A shows the spectra (Ex=414 nm) from one set of experiments, in which the CyPetRanGAP1c concentration was fixed to 1 μM. As the concentration of YPetUbc9 was gradually increased from 0 to 4 μM, binding of YPetUbc9 to CyPetRanGAP1c resulted in an energy transfer from CyPet to YPet, and the emission at 530 nm was significantly increased, while the direct emission at 475 nm of CyPetRanGAP1c was decreased. It was then determined the fluorescence emissions of each component in four different concentrations of CyPetRanGAP1c. In these six sets of experiments, the concentration of CyPetRanGAP1c was fixed to 0.05, 0.1, 0.5, 1.0 μM, and the concentration of YPetUbc9 was increased from 0 to 4 μM in each assay. The fluorescence emission spectra of all mixtures were then determined at the excitation wavelengths of 414 and 495 nm to exclude the direct emissions of CyPetRanGAP1c and YPetUbc9. After subtracting the direct emissions of CyPetRanGAP1c and YPetUbc9, the absolute FRET emission (at 530 nm when Ex=414 nm) increased steadily when more YPetUbc9 was added at each concentration of CyPetRanGAP1c (see FIG. 3A).

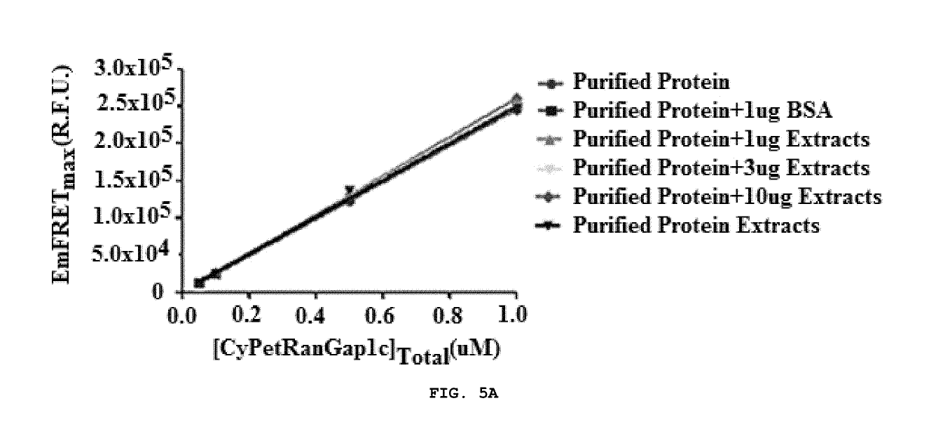

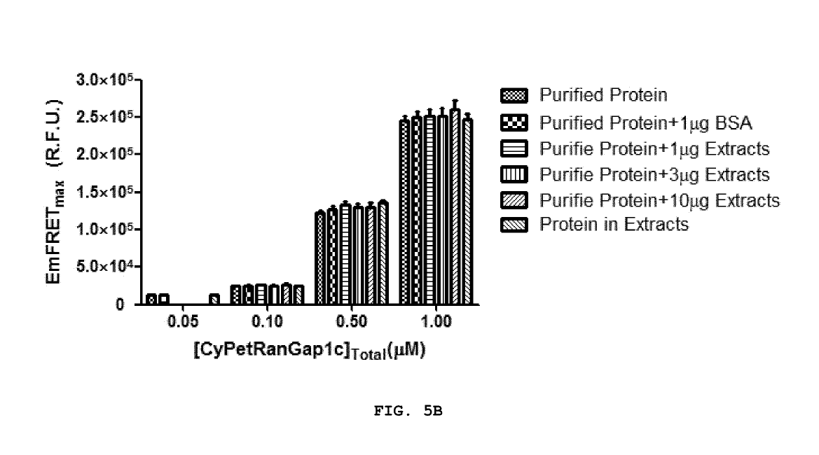

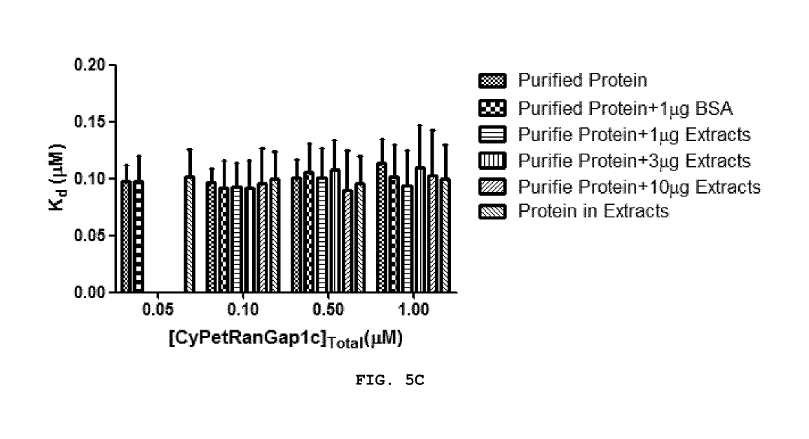

To determine Em_{FRETmax} and K_{d}'s, Equation (6) was applied in each set of experiments with different total concentrations of CyPetRanGAP1c using nonlinear regression. After the above calculations were used to determine the direct emissions of CyPetRanGAP1c and YPetUbc9 at 530 nm, the FRET signals (Em_{FRET}) were obtained by subtracting the direct emission of CyPetRanGAP1c (α*FL_{DD}) and YPetUbc9 (β*FL_{AA}) at 530 nm from the total emission at 530 nm (FL_{DA}). So from the nonlinear regression in Prism 5, the estimated values for Em_{FRETmax}, were (1.227±0.022)×10⁴, (2.433±0.041)×10⁴, (12.29±0.23)×10⁴, and (24.61±0.53)×10⁴, for 0.05, 0.1, 0.5, 1.0 μM of CyPetRanGAP1c, respectively. In this concentration range of the binding partner, the Em_{FRETmax} had a linear relationship with CyPetRanGAP1c from 3 to 60 pmole (R²=1.000, see FIGS. 5A and 5B and Table 1). This result suggests that the approach accurately and consistently predicted Em_{FRETmax} at various concentration ratios of CyPetRanGAP1c and YPetUbc9.

TABLE 1
Summary of the maximal FRET emission Em_{FRETmax}
and K_{d} values in different conditions.

| | | Em_{FRETmax} |
|---|---|---|
| CyPetRanGAP1c (μM) | Kd (μM) | (R.F.U.) × 10⁴ |
| 0.05 | 0.098 ± 0.014 | 1.227 ± 0.022 |
| 0.1 | 0.096 ± 0.013 | 2.433 ± 0.041 |
| 0.5 | 0.101 ± 0.016 | 12.29 ± 0.23 |
| 1 | 0.114 ± 0.021 | 24.61 ± 0.53 |
| 0.05 + 1 μgBSA | 0.098 ± 0.022 | 1.260 ± 0.036 |
| 0.1 + 1 μg BSA | 0.092 ± 0.024 | 2.523 ± 0.080 |
| 0.5 + 1 μg BSA | 0.105 ± 0.025 | 12.71 ± 0.38 |
| 1.0 + 1 μg BSA | 0.102 ± 0.028 | 24.97 ± 0.76 |
| 0.1 + 1 μg bacterial extracts | 0.092 ± 0.022 | 2.583 ± 0.074 |
| 0.1 + 3 μg bacterial extracts | 0.092 ± 0.023 | 2.572 ± 0.079 |
| 0.1 + 10 μg bacterial extracts | 0.096 ± 0.031 | 2.636 ± 0.106 |
| 0.5 + 1 μg bacterial extracts | 0.100 ± 0.027 | 13.26 ± 0.43 |
| 0.5 + 3 μg bacterial extracts | 0.108 ± 0.025 | 13.02 ± 0.39 |
| 0.5 + 10 μg bacterial extracts | 0.090 ± 0.035 | 13.07 ± 0.58 |
| 1.0 + 1 μg bacterial extracts | 0.093 ± 0.031 | 25.21 ± 0.90 |
| 1.0 + 3 μg bacterial extracts | 0.109 ± 0.037 | 25.19 ± 0.99 |
| 1.0 + 10 μg bacterial extracts | 0.103 ± 0.040 | 26.06 ± 1.14 |
| 0.05 (from crude extract) | 0.102 ± 0.024 | 1.308 ± 0.041 |
| 0.1 (from crude extract) | 0.100 ± 0.024 | 2.447 ± 0.075 |
| 0.5 (from crude extract) | 0.096 ± 0.023 | 13.57 ± 0.39 |
| 1.0 (from crude extract) | 0.100 ± 0.029 | 24.63 ± 0.79 |
| SPR Method by Ling | 0.097 | |
| 0.05 | 0.098 ± 0.014 | 1.227 ± 0.022 |
| 0.1 | 0.096 ± 0.013 | 2.433 ± 0.041 |
| 0.5 | 0.101 ± 0.016 | 12.29 ± 0.23 |
| 1 | 0.114 ± 0.021 | 24.61 ± 0.53 |
| 0.05 + 1 μgBSA | 0.098 ± 0.022 | 1.260 ± 0.036 |
| 0.1 + 1 μg BSA | 0.092 ± 0.024 | 2.523 ± 0.080 |
| 0.5 + 1 μg BSA | 0.105 ± 0.025 | 12.71 ± 0.38 |
| 1.0 + 1 μg BSA | 0.102 ± 0.028 | 24.97 ± 0.76 |
| 0.1 + 1 μg bacterial extracts | 0.092 ± 0.022 | 2.583 ± 0.074 |
| 0.1 + 3 μg bacterial extracts | 0.092 ± 0.023 | 2.572 ± 0.079 |
| 0.1 + 10 μg bacterial extracts | 0.096 ± 0.031 | 2.636 ± 0.106 |
| 0.5 + 1 μg bacterial extracts | 0.100 ± 0.027 | 13.26 ± 0.43 |
| 0.5 + 3 μg bacterial extracts | 0.108 ± 0.025 | 13.02 ± 0.39 |
| 0.5 + 10 μg bacterial extracts | 0.090 ± 0.035 | 13.07 ± 0.58 |
| 1.0 + 1 μg bacterial extracts | 0.093 ± 0.031 | 25.21 ± 0.90 |
| 1.0 + 3 μg bacterial extracts | 0.109 ± 0.037 | 25.19 ± 0.99 |
| 1.0 + 10 μg bacterial extracts | 0.103 ± 0.040 | 26.06 ± 1.14 |
| 0.05 (from crude extract) | 0.102 ± 0.024 | 1.308 ± 0.041 |
| 0.1 (from crude extract) | 0.100 ± 0.024 | 2.447 ± 0.075 |
| 0.5 (from crude extract) | 0.096 ± 0.023 | 13.57 ± 0.39 |
| 1.0 (from crude extract) | 0.100 ± 0.029 | 24.63 ± 0.79 |
| SPR Method by Ling | 0.097 | |

The disassociation constants of CyPetRanGAP1c and YPetUbc9 were then determined from non-linear regression. By plotting (Eq. 6) with Em_{FRET} vs. [YPetUbc9]ₜₒₜₐₗ in the Prism 5 program, the K_{d}s were determined from four concentrations of CyPetRanGAP1c (0.05, 0.1, 0.5, and 1.0 μM) as 0.098±0.014, 0.096±0.013, 0.101±0.016, and 0.114±0.021 μM, respectively (see Table 1). The very close K_{d}s generated from different concentrations of CyPet-RanGAP1c (from 0.05 μM to 1.0 μM of CyPetRanGAP1c) and a small binding partner ratios of CyPetRanGAP1c to YPetUbc9 (from 0.67 to 40 fold), demonstrate that the FRET-based K_{d} measurement approach disclosed herein is very robust and reliable.

Em_{FRET} Determination in the Presence of BSA and Bacterial Extracted Proteins.

To order to validate the assumption that the FRET-based K_{d} determination method of the disclosure can be used for measuring K_{d} in the presence of a third protein or in the presence of contaminated proteins, the fluorescence emissions were determined for each component in three different concentrations of CyPetRanGAP1c, in the presence of BSA or bacterial contaminated proteins. In these three sets of experiments, the concentration of CyPetRanGAP1c was fixed to 0.05, 0.1, 0.5 and 1.0 μM; the concentration of YPetUbc9 was increased from 0 to 4 μM; the amount of BSA was 1 μg; and the bacterial contaminated proteins were used at 1, 3, 10 μg.

To determine Em_{FRETmax} and K_{d}'s, (Eq. 6) was applied in each set of experiments with different total concentrations of CyPetRanGAP1c using nonlinear regression. After the above calculations were used to determine the direct emissions of CyPetRanGAP1c and YPetUbc9 at 530 nm, the FRET signals (Em_{FRET}) were obtained by subtracting both the direct emission of CyPetRanGAP1c (α*FL_{DD}), YPetUbc9 (β*FL_{AA}) at 530 nm from the total emission at 530 nm (FL_{DA}). Additionally, the BSA and contaminate proteins background were determined and subtracted.

The disassociation constants of CyPetRanGAP1c and YPetUbc9 in the presence of BSA were determined from the nonlinear regression. By plotting (Eq. 6) with Em_{FRET} vs. [YPetUbc9]ₜₒₜₐₗ in the Prism 5 program, the K_{d}s from three concentrations of CyPetRanGAP1c (0.05, 0.1, 0.5, and 1.0) were determined as 0.098±0.022, 0.092±0.024, 0.105±0.025, 0.102±0.028 μM. The average K_{d} is 0.099±0.006, compared to K_{d} without BSA 0.102±0.008 μM. The results only changed by a little amount.

The disassociation constants, K_{d}s, of CyPetRanGAP1c and YPetUbc9 in the presence of bacterial contaminated proteins were determined from three concentrations of CyPetRanGAP1c. When the concentration of CyPetRanGAP1c was fixed as 0.1 μM, 1.0, 3.0, 10.0 μg, bacterial contaminated proteins were added to the mixture. The K_{d}s were 0.092±0.022, 0.092±0.023, 0.096±0.031 μM, respectively. When the concentration of CyPetRanGAP1c was fixed as 0.5 μM, 1.0, 3.0, 10.0 μg, the bacterial contaminated proteins were added to the mixture. The K_{d}s were 0.100±0.027, 0.108±0.025, 0.090±0.035 μM, respectively. When the concentration of CyPetRanGAP1c was fixed as 1.0 μM, 1.0, 3.0, 10.0 μg, bacterial contaminated proteins were added to the mixture. The K_{d}s were 0.093±0.031, 0.109±0.037, 0.103±0.040 μM, respectively. Under these two conditions, Em_{FRET max} was very stable (see FIG. 5A-B; see also Table 1).

The very consistent and accurate results presented herein suggest that the dissociation constant, K_{d}, can be calculated by the quantitative FRET assay using the methods described herein even in the absence or presence of BSA and bacterial contaminated proteins.

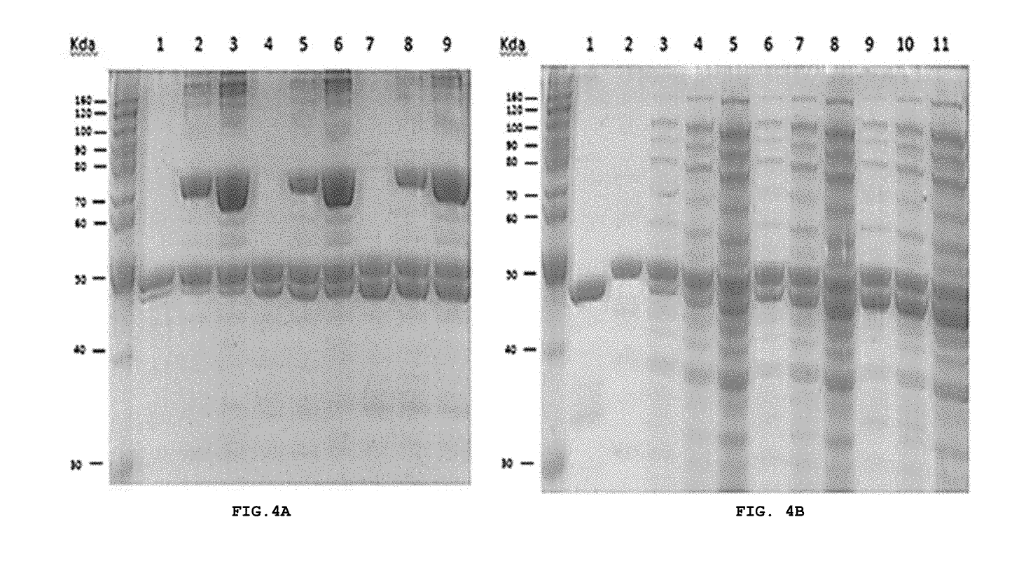

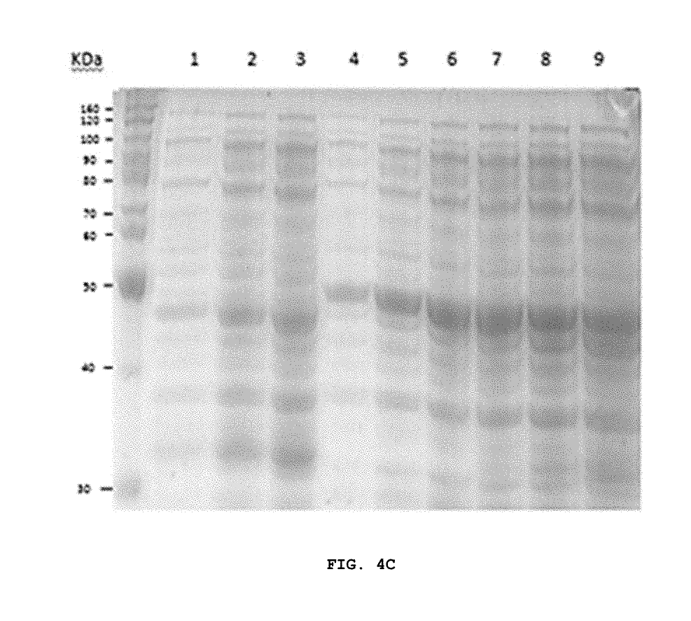

By using commassie blue staining of polyacrylamide gels, it was determined that the molecular weights of CyPetRanGAP1c and YPetUbc9 is 47.4 KDa and 47.6 KDa, respectively (see FIG. 4B). From the gel, lanes 1 and 2 of FIG. 4B, are CyPetRanGAP1c and YPetUbc9 purified from bacterial lysates using nickel agarose affinity chromatography. Lanes 2, 3, 5, 6, 8 and 9 are a pure mixture of CyPetRanGAP1c and YPetUbc9 with 1 μg or 3 μg of BSA added (see FIG. 4A). From the gel of FIG. 4B, lane 1, 2 are pure CyPetRanGAP1c and YPetUbc9, 3˜11 are a pure mixture of CyPetRanGAP1c and YPetUbc9 with 1 μg, 3 μg or 10 μg of bacterial lysate proteins (see FIG. 4C).

Em_{FRETmax} and K_{d} Determinations in the Presence of Other Proteins.

The values of Em_{FRETmax} for mixtures comprising CyPetRanGAP1c and YPetUbc9 in the presence of BSA, were (1.260±0.036)×10⁴, (2.523±0.080)×10⁴, (12.71±0.38)×10⁴ and (24.97±0.76)×10⁴, for 0.05, 0.1, 0.5, 1.0 μM of CyPetRanGAP1c, respectively (see Table 1). The Em_{FRET max} exhibited a linear relationship and the curve overlapped with the one without BSA. The graphs also show that Em_{FRETmax} is stable after 1 μg BSA is added (see FIG. 5A-B). The values of Em_{FRETmax} for mixtures comprising CyPetRanGAP1c and YPetUbc9 in the presence of 1 μg of E. coli lysates were 2.583±0.074, 13.26±0.43, and 25.21±0.90, respectively; 2.572±0.079, 13.02±0.39, and 25.19±0.99 when 3 μg of E. coli lysates as added; and 2.636±0.106, 13.07±0.58, and 26.06±1.14 when 10 μg of E. coli lysate was added (see Table 1). The results when compared, demonstrate that the value of K_{d} or Em_{FRET max} had a non-significant change despite the changing protein mixtures. For example, the same K_{d} and EmFRET max value was obtained for a mixture of CyPetRanGAP1c and YPetUbc9 with no contaminate proteins, a mixture of CyPetRanGAP1c and YPetUbc9 with one contaminate protein, and a mixture of CyPetRanGAP1c and YPetUbc9 comprising multiple contaminate proteins. Accordingly, the FRET-based method disclosed herein can be used to determine K_{d} from complicated assay conditions. Accordingly, the methods disclosed herein allow for the study of protein interactions which heretofore have not been previously studied.

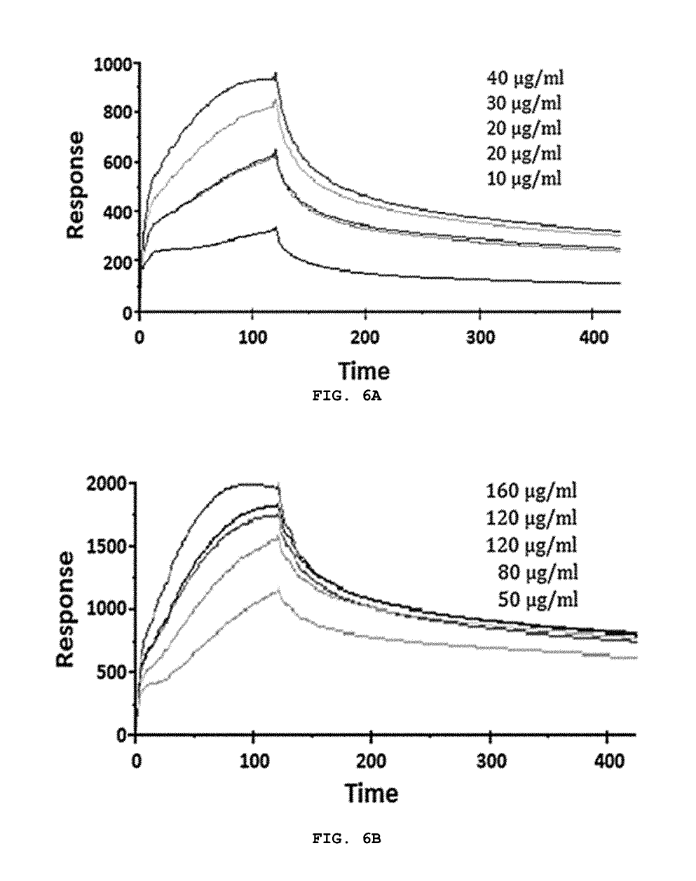

To validate the K_{d} determinations from the FRET assay, the interaction disassociation constant was studied for CyPetRanGAP1c and YPetUbc9 by SPR. His-tagged YPetUbc9 and CyPetRanGAP1c were expressed in bacterial cells and purified using Ni-NTA agarose beads. After dialysis against Biacore running buffer, His-tagged YPetUbc9 was immobilized onto a SPR NTA sensor chip. The CyPetRanGAP1c was obtained by cleaving the His-tag on nickel beads directly by thrombin digestion. Non-specific binding of CyPetRanGAP1c to the NTA chip was subtracted by response signal of control channel of the NTA sensor chip. The binding response of the bound YPetUbc9 to injections of a series of concentrations of the CyPetRanGAP1c was studied by a binding kinetics analysis (see FIG. 6A). The CyPetRanGAP1c bound with moderate kinetics to the YPetUbc9, with a calculated K_{d} of 0.097 μM. This K_{d} was close to these K_{d}s determined by the quantitative FRET methods described above. To further analyze the possible interference of fluorescence tag to the interaction, a control experiment was performed with untagged proteins in SPR. The interaction of RanGAP1c and Ubc9 by themselves was analyzed. Similar to above experiment, His-tagged Ubc9 was immobilized onto NTA sensor chip and RanGAP1c was flow phase. The binding response of bound Ubc9 to a series of different RanGAP1c concentrations show similar kinetics as the fluorescent protein-tagged fusion proteins (see FIG. 6B). The K_{d} of the RanGAP1c and Ubc9 interaction was calculated as 0.182 μM, which is consistent with that of the fluorescent protein-tagged RanGAP1c and Ubc9.

K_{d} Determination from Fluorescent Proteins in Bacterial Crude Extracts without Purification.

The CyPetRanGAP1c and YPetUbc9 proteins look very dity (see FIG. 5 C). Both CyPetRanGAP1c and YPetUbc9 are easy to differentiate because their expression levels are high. Although it is hard to determine K_{d} under these conditions using other methods, the method of the disclosure allow for such a determination. The real concentration of CyPetRanGAP1c and YPetUbc9 in the crude extracts can be determined by monitoring the fluorescence signal at 475 nm and 530 nm from standard curve, respectively. The concentration of CyPetRanGAP1c was fixed at 0.05, 0.1, 0.5, 1.0 μM; the concentration of YPetUbc9 was increased from 0 to 4 μM. The K_{d}s were found to be 0.102±0.024, 0.100±0.024, 0.096±0.023 and 0.100±0.029 μM, respectively. This result was surprisingly stable and fully agreed with the results using pure proteins (see Table 1). The Em_{FRETmax} was still linear as well, (1.308±0.041)×10⁴, (2.447±0.075)×10⁴, (13.57±0.39)×10⁴ and (24.63±0.79)×10⁴. Both Em_{FRET max} and K_{d}s did not change when compared with the results from the pure protein assays.

A number of embodiments have been described herein. Nevertheless, it will be understood that various modifications may be made without departing from the spirit and scope of this disclosure. Accordingly, other embodiments are within the scope of the following claims.

1. A Forester Resonance Energy Transfer (FRET)-based molecule interaction method to determine a dissociation constant (K_{d}) between two molecules in the presence of one or more contaminant molecules, comprising:
providing a mixture comprising a first molecule comprising a FRET donor and a second molecule comprising a FRET acceptor, wherein the mixture may further comprise one or more contaminant molecules;
determining the absolute FRET emission signal value (Em_{FRET});
determining the maximum amount of the FRET-donor/first molecule is bound by FRET-acceptor/second molecule (EM_{FRETmax}) by using nonlinear regression and measuring the emissions from fixed concentrations of FRET donor/first molecule and varying concentrations of the FRET acceptor/second molecule by exciting the FRET pair; and
determining the K_{d} for the first and second molecule, by using nonlinear regression and the formula of:
$${Em}_{FRET} = {Em}_{{FRET}_{max}} ( 1 - \frac{2 {K}_{d}}{X - A + {K}_{d} + \sqrt{{( X - A - {K}_{d} )}^{2} + 4 {K}_{d} X}} )$$
wherein, A is the total concentration of FRET donor/first molecule, and X is the total concentration of FRET acceptor/second molecule.

2. The method of claim 1, wherein EM_{FRET} can be determined using the formula of:
Em_{FRET}=FL_{DA}−α*FL_{DD}−β*FL_{AA}
wherein, FL_{DA} is the fluorescence emission that is measured when FRET donor is excited by a first wavelength of light and transmits the energy to the FRET acceptor, FL_{DD} is the fluorescence emission of FRET donor/first molecule when excited by a second wavelength of light, and FL_{AA} is the fluorescence emission of FRET acceptor/second molecule when excited by a third wavelength of light, α is constant determined by using free FRET donor/first molecule, and β is a constant determined by free using FRET acceptor/second molecule.

3. The method of claim 1, wherein the FRET donor is CyPet or other FRET donors.

4. The method of claim 1, wherein the FRET acceptor is YPet or other FRET acceptors.

5. The method of claim 1, wherein the first and second molecule are independently selected from the group consisting of a peptide, a polypeptide, a protein, a nucleic acid molecule, a lipid, and a polysaccharide.

6. The method of claim 1, wherein the first molecule and second molecule are an enzyme and its substrate.

7. The method of claim 1, wherein the first molecule and second molecule are a receptor and its ligand.

8. The method of claim 1, wherein the first molecule and second molecule are an antibody and its antigen.

9. The method of claim 1, wherein the first molecule and second molecule are a protein and its interacting partner(s).

10. The method of claim 2, wherein the first wavelength of light is between 400 to 800 nm.

11. The method of claim 1, wherein the first molecule comprising a FRET donor comprises a fusion protein.

12. The method of claim 1, wherein the second molecule comprising a FRET acceptor comprises a fusion protein.

13. The method of claim 1, wherein the first molecule and second molecule are expressed in the same cell.

14. The method of claim 12, wherein the K_{d} is determine in an intact cell.

15. The method of claim 12, wherein the K_{d} is determined in a disrupted cell preparation.

16. The method of claim 1, wherein the first and second molecule are expressed and isolated and mixed with contaminant molecules.

17. The method of claim 1, wherein the first molecule comprising a FRET donor comprises an engineered protein.

18. The method of claim 1, wherein the second molecule comprising a FRET acceptor comprises an engineered protein.

19. The method of claim 1, wherein the first or second molecule comprise DNA.

20. The method of claim 1, wherein the first or second molecule comprise lipids.

21. The method of claim 1, wherein the first or second molecule comprise polysaccharides.
